(19) United States
(12) Patent Application Publication
(10) Pub. No.: US 2025/0195742 A1
(43) Pub. Date: Jun. 19, 2025

(54) APPARATUS FOR NEGATIVE PRESSURE WOUND THERAPY

(71) Applicant: BRIGHTWAKE LIMITED, Nottinghamshire (GB)

(72) Inventors: Stephen COTTON, Nottinghamshire (GB); Matthew TASKER, Nottinghamshire (GB)

(21) Appl. No.: 18/846,408

(22) PCT Filed: Dec. 19, 2023
(86) PCT No.: PCT/GB2023/053306

§ 371 Date: Sep. 12, 2024

(30) Foreign Application Priority Data
Dec. 22, 2022 (GB) 2219605.9

(51) Int. Cl. A61M 1/00

(52) U.S. Cl. CPC A61M 1/94; A61M 1/915; A61M 1/966

(57) ABSTRACT

A device (1) for negative pressure wound therapy (NPWT) comprising a source of negative pressure (2) and a single gas reservoir (4) in fluid communication with the source of reduced pressure (2), the single gas reservoir (2) comprising means for establishing fluid communication with a wound; a system for NPWT comprising the device (1) and at least one wound dressing (14) for NPWT; and a kit comprising the device (1) and a plurality of dressings for NPWT.

The present invention relates to a device for negative pressure wound therapy (NPWT), and more specifically to a NPWT device including a source of reduced pressure and a gas reservoir.

### BACKGROUND TO THE INVENTION

Negative pressure wound therapy (NPWT) involves the application of a pressure that is reduced relative to the surroundings (commonly referred to as “negative pressure”) to a wound, which causes mechanical contraction of the wound and removal of wound fluid from the wound, thus promoting formation of granulation tissue and accelerating wound healing. The technique is particularly effective in the treatment of slow healing wounds such as chronic leg ulcers and large open wounds. An occlusive dressing is applied to the wound and forms a seal around the wound under which a negative pressure can be established. The occlusive dressing is traversed by a drainage tube that is connected to a source of negative pressure, enabling the application of reduced pressure to the wound and the drainage of wound fluid from the wound. The negative pressure that is applied to a wound is typically around 100 mmHg, although a negative pressure in the range of 80-120 mmHg is usually tolerated as movement of the patient or direct pressure on the wound can cause the pressure at the wound site to fluctuate.

NPWT systems for single patient use are known. Such systems are indicated for patients with low to moderately exuding wounds, including leg ulcers and other chronic wounds, acute wounds, subacute and dehisced wounds, traumatic wounds, flaps and grafts, and surgically closed incision sites. A typical system may comprise a wound dressing connected to a small, generally disposable and battery-operated pump that delivers the NPWT. When the pump is switched on, negative pressure is applied to the dressing and wound fluid is absorbed into the dressing. A pressure sensor monitors the negative pressure at the wound, and when the desired negative pressure is reached, feeds back to the pump to cease the application of negative pressure.

One such system is the Avelle™ negative pressure wound therapy system, by Convatec, Flintshire, UK.

One issue with existing commercial NWPT systems for single patient use is that the speed at which negative pressure is delivered to the wound dressing is such that the negative pressure can exceed the desired level in the interval between the pressure sensor detecting that the desired level of negative pressure has been reached and the pump ceasing application of negative pressure. This additional undesired, application of negative pressure not only wastes energy, but can have negative effects on the patient. For example, excess pressure can cause pain and discomfort to the patient

GB 2560365 discloses an apparatus for NWPT comprising a vacuum pump and first and second chambers. The first chamber is in fluid communication with the pump, while the second chamber has a port for fluid connection with a wound. In operation, activation of the vacuum pump generates a negative pressure in the first chamber, which negative pressure is prevented from exceeding −120 mmHg by means of a pressure release valve that permits entry of gas into the first chamber from the surroundings. The generation of a negative pressure in the first chamber generates a pressure differential between the first and second chambers, resulting in the passage of gas from the second chamber into the first chamber via a one-way valve between the two chambers and the generation of a negative pressure (which cannot exceed −120 mmHg) in the second chamber, which is in direct fluid communication with the wound. A pressure sensor detects the negative pressure in the second chamber and deactivates the pump when a predetermined negative pressure is reached.

### BRIEF SUMMARY OF THE INVENTION

The present inventors have now devised an improved device for negative pressure wound therapy. The device comprises a source of reduced pressure and a single gas reservoir, which single gas reservoir is in fluid communication with the source of reduced pressure and comprises means for establishing fluid communication with a wound. The device forms a first aspect of the present invention.

No other gas reservoir is present in the device.

The device may be a single-patient use device, which may be disposable and which may be, but is not necessarily, battery operated.

The device of the first aspect of the present invention may be used in a system for negative pressure wound therapy as described herein, which system forms a second aspect of the present invention.

A kit for negative pressure wound therapy as described herein forms a third aspect of the present invention.

A method of negative pressure wound therapy as described herein forms a fourth aspect of the present invention.

The inclusion of a single gas reservoir in the device of the present invention is advantageous at least in that the time interval between operation of the source of reduced pressure and the application of reduced pressure at the wound site is longer than it is with conventional, commercially available devices. This allows sufficient time for a pressure sensor to indicate that the desired negative pressure has been reached and to feed back to the source of reduced pressure to cease operation. In addition, having a single gas reservoir, the device is robust, easy to manufacture, and not prone to leakage.

### DETAILED DESCRIPTION

### Device for Negative Pressure Wound Therapy (NPWT)

In accordance with a first aspect, the present invention provides a device for NPWT, comprising a source of negative pressure and a single gas reservoir, which single gas reservoir is in fluid communication with the source of negative pressure and comprises means for establishing fluid communication with a wound.

The device may further comprise means for detecting the negative pressure that is applied to the wound, which means may be operably coupled to the source of reduced pressure, for example via a programmable computer processor comprised in the device.

The device may be, but is not necessarily, battery operated. It may be, but is not necessarily, for single-patient use, and may be, but is not necessarily, disposable.

The source of reduced pressure is most suitably a vacuum pump. For example, the source of reduced pressure may be a diaphragm vacuum pump, an electromagnetic vacuum pump, or a piezoelectric vacuum pump. The source of reduced pressure may be compact and for example may have a maximum dimension of about 100 mm or less, about 80 mm or less, about 60 mm or less, or about 40 mm or less. It may occupy a volume of about 10 cm³ or less, about 8 cm³ or less, about 6 cm³ or less, or about 4 cm³ or less. One example of a vacuum pump that is suitable for use with this invention is the KPV08A-3A Micro Vacuum Pump by Koge Micro Tech Co Ltd. The KPV08A-3A Micro Vacuum Pump is a diaphragm pump. The terms ‘source of reduced pressure’ and ‘pump’ are used interchangeably herein.

The pump may be housed within a manifold. Housing the pump within a manifold reduces noise. The manifold may comprise a relatively soft material at least in the regions in contact with other components of the device, in order to facilitate the assembly of the components of the device and provide good surface area contact and an effective seal between components. The manifold may be formed entirely of the relatively soft material. Suitable materials include thermoplastic elastomers such as thermoplastic polyurethane (TPU) and equivalent materials such as will be known to the person skilled in the art.

The single gas reservoir may be at least partly housed within the manifold. For example, the single gas reservoir may be housed in the manifold to an extent desirable for connection and sealing thereto. The single gas reservoir may comprise a hard material. Suitable materials include acrylonitrile-butadiene-styrene (ABS) and equivalent materials such as will be known to the person skilled in the art. The material of the reservoir is a hard material.

The single gas reservoir is in fluid communication with the pump, for example via a vacuum port located on the pump. Preferably, a check valve is located between the pump and the single gas reservoir. The check valve may be, for example, a one-way valve that permits the flow of gas out of the single gas reservoir but that does not allow a flow of gas into the single gas reservoir.

Examples of suitable one-way valves are diaphragm valves, ball and spring valves and swing/tilt valves. One valve that is suitable for use in the present device is the SCV 67220 Barbed Check Valve by Nordson Corporation.

The single gas reservoir comprises means for establishing fluid communication with a wound. Said means may comprise a connector, such as a Luer lock or another conventional medical connector. The means preferably permits the formation of an air-tight or substantially air-tight connection between the single gas reservoir and the wound, for example via suction tubing that is adapted to be connected with a wound dressing. Suction tubing extending from the device May be connected directly to a wound dressing, or it may be connected to a connector, for example a Y-piece; in the latter case, each of two additional lengths of suction tubing may be connected at one end to the Y-piece and at the other end to a wound dressing for NPWT.

The device may comprise means for detecting the negative pressure that is applied to the wound. Said means may detect the negative pressure in the single gas reservoir, as the single gas reservoir and the wound are in fluid communication and are thus at substantially the same negative pressure during operation of the device. The means for detecting the negative pressure that is applied to the wound may be in fluid communication with the single gas reservoir.

The means for detecting the negative pressure that is applied to the wound may be operably coupled to the pump, for example such that the pump is deactivated in response to the negative pressure that is applied to the wound reaching a desired level and/or such that the pump is activated in response to the negative pressure that is applied to the wound dropping below a desired level. This arrangement may enable the device to maintain the negative pressure that is applied to the wound at a desired level for as long as the device is in operation, without the need for further intervention by an operator.

The desired level of negative pressure that is applied to the wound may be from about 60 mm Hg to about 125 mmHg, and is preferably from about 70 mm Hg to about 90 mmHg, such as about 80 mmHg. Thus, the programmable computer processor may be programmed to deactivate the pump when the negative pressure detected by the pressure sensor reaches a predetermined maximum level following activation, and to reactivate the pump when the negative pressure falls to a predetermined minimum level. For example, the predetermined maximum level of negative pressure may be from about 90 mmHg to about 125 mmHg, such as about 90 mmHg, about 95 mmHg, about 100 mmHg, about 105 mmHg, about 110 mmHg, about 115 mmHg, about 120 mmHg or about 125 mmHg. The predetermined minimum level of negative pressure may be from about 70 mmHg to about 60 mmHg, such as about 70 mmHg, about 65 mmHg, or about 60 mmHg.

The means for detecting the negative pressure that is applied to the wound may be an electronic pressure sensor such as a piezoresistive electronic pressure sensor. One pressure sensor that is suitable for use with this invention is the 2SMPP-03 MEMS Gauge Pressure Sensor by Omron Corporation.

Operable coupling of the means for detecting the negative pressure that is applied to the wound to the pump may be via a programmable computer processor that permits the desired level of negative pressure that is applied to the wound to be varied, which programmable computer processor may be comprised in the device. For example, pressure sensor may be operably coupled to the pump via a printed circuit board (PCB) comprised in the device. The pressure sensor may be embedded in the PCB. The pump may be secured to the PCB.

The programmable computer processor may comprise a power button and LED (light emitting diode) units or other detection and display means (collectively, ‘indicators’) for indicating whether the pump is on or off, if there is a leak in the system, and/or if the batteries need to be replaced.

The device may also comprise wireless capability, for example Bluetooth capability, for example in the form of a Bluetooth chip.

The device may further comprise a housing that accommodates the other components of device as described herein, and which optionally comprises a battery compartment. The housing may comprise or be formed of ABS or an equivalent material, such as will be known to the person skilled in the art.

The housing may comprise a power button that is operably coupled to the pump, for example via the power button on the programmable computer processor. The housing may further comprise transparent windows, for example clear plastic lenses, through which the LED or other indicators on the programmable computer processor are visible.

Preferably, the device is disposable. In particular, the device may be programmed to cease functioning after a set period of time, such as 60 days, 45 days or 30 days from the time of the first operation of the device.

Preferably, the maximum power consumption of the device is less than 5 W, less than 3 W, or less than 2 W.

The single gas reservoir, manifold and housing of the present device may be manufactured using a moulding process such as injection moulding. The device may then be assembled by hand.

### System for NPWT

In accordance with a second aspect, the present invention provides a system for negative pressure wound therapy, which system comprises a device for NPWT as described herein and a wound dressing for NPWT.

Wound dressings for NWPT are known and any wound dressing for NPWT is suitable for use in the present invention. A particularly suitable dressing is described in WO2022049363, which is incorporated herein by reference, in its entirely.

The wound dressing for use in the present invention may be an island wound dressing as described in WO2022049363, comprising a backing layer, an absorbent pad and a wound contacting layer, the absorbent pad being disposed between the backing layer and the wound contacting layer and the backing layer comprising an aperture which brings the absorbent pad into fluid communication with the exterior of the wound dressing.

The wound contacting layer may be a layer of silicone gel, more preferably a layer of perforated silicone gel. The silicone gel may be a hydrophobic silicone gel.

The aperture may be located directly above or on top of the absorbent pad. The dressing may be supplied with the aperture occluded by a releasable liner, which may be made of siliconized paper or another suitable material. The aperture may be any shape suitable for connection to a suction tube, but it is preferably circular. The aperture is of a size to enable it to be connected to a suction tube, and typically has a diameter of from about 5 mm to about 20 mm, or from about 8 mm to about 15 mm, or about 10 mm to about 14 mm.

The dressing may have a moisture-permeable layer of pressure-sensitive adhesive comprising, for example, rubber/resin adhesive, acrylic adhesive or acrylate adhesive, disposed between the backing layer and the absorbent pad, or between the absorbent pad and the wound contacting layer.

The system of the present invention is not limited to the dressing described above.

Typically, the wearable time of a dressing for NPWT is around 3-4 days, and generally up to a maximum of 7 days. For example, the dressing will need to be changed when its absorption capacity has been reached, and if the port becomes blocked with exudate. Thus, the device may be supplied in kit form, with multiple dressings.

### Kit for NPWT

In accordance with a third aspect of the present invention, there is provided a kit for negative pressure wound therapy (NPWT), which kit comprises a device for NPWT as described herein, and a plurality of wound dressings for NPWT.

The plurality of wound dressings for NPWT may comprise island wound dressings as described in WO2022049363, and above.

In addition to the device of the present invention and a plurality of wound dressings for NPWT, the kit in accordance with this aspect of the invention may comprise additional components, for example, any of batteries, suction tubing, a Y-piece or other connector, and sealing strips for attaching a dressing for NPWT to the skin of a patient.

### Method for NPWT

In accordance with a fourth aspect of this invention, there is provided a method of negative pressure wound therapy, comprising applying a wound dressing for negative pressure wound therapy (NPWT) to a wound; establishing fluid communication between the wound dressing and the single gas reservoir of the device as described herein; and activating the device.

For example, a wound dressing for NPWT may be applied over a wound and one end of a suction tube secured to the dressing. The other end of the suction tube may be secured to the device to provide means for fluid communication between the single gas reservoir and the wound. The device may then be activated, such that negative pressure is applied to the wound. No further manual input is required by the user or operator when the device comprises a pressure sensor coupled to a programmable computer processor to detect the negative pressure and operate or deactivate the pump in response to the measured negative pressure, unless the dressing needs to be changed, a leak in the system is detected, or the batteries need to be replaced.

The method may be used with multiple, for example two, wound dressings for NPWT, each connected to a suction tube that is secured to a Y-piece or another connector from which further tubing connected thereto extends and connects to the device.

The wound dressing for NPWT may comprise an island wound dressing as described in WO2022049363, and above.

### FIGURES

FIG. 1 is an exploded view of the internal arrangement of a device in accordance with an embodiment of the first aspect of the present invention.

FIG. 2 is an assembled view of the internal arrangement of the device illustrated in FIG. 1.

FIG. 3 is a reverse view of the device illustrated in FIG. 2.

FIG. 4 illustrates a housing in accordance with an embodiment of the present invention.

FIG. 5 illustrates a device in accordance with an embodiment of the present invention, connected to two dressings for NPWT.

### DESCRIPTION OF A SPECIFIC EMBODIMENT AND METHOD OF USE

The present invention is not limited to the specific embodiment described herein, which is for illustrative purposes. The specific features described below may be combined with any other part of the above description.

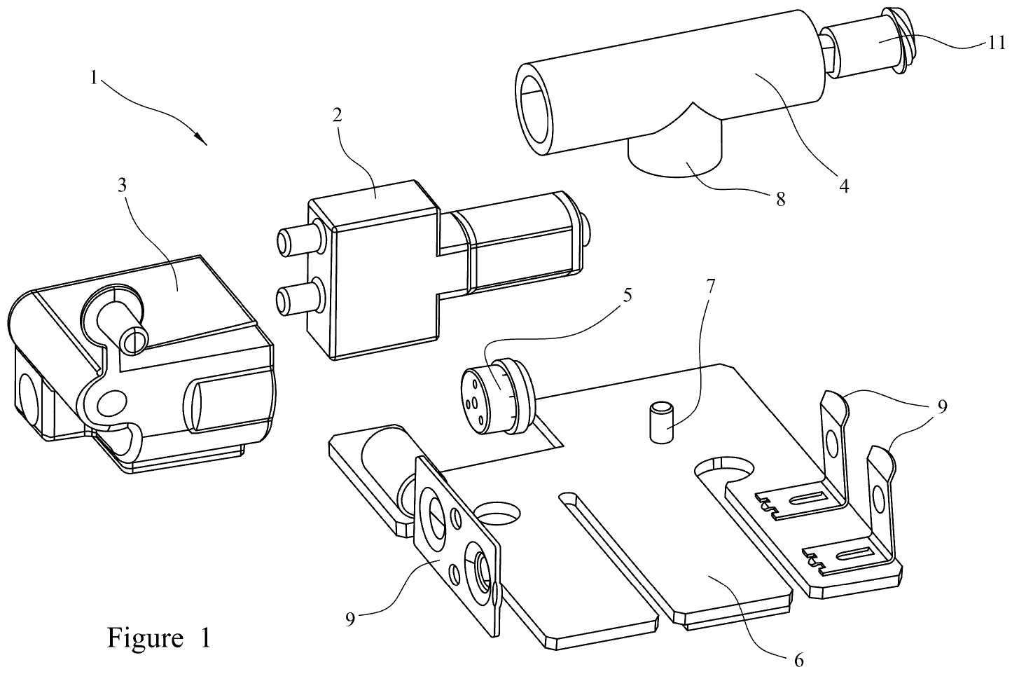

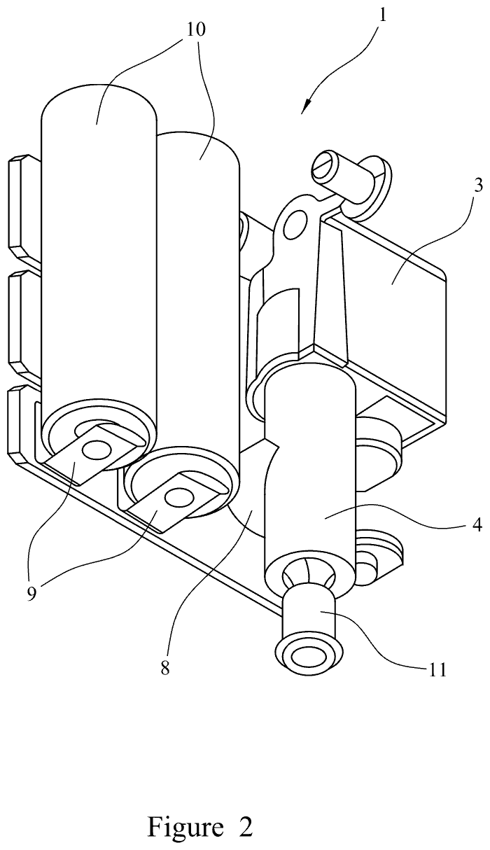

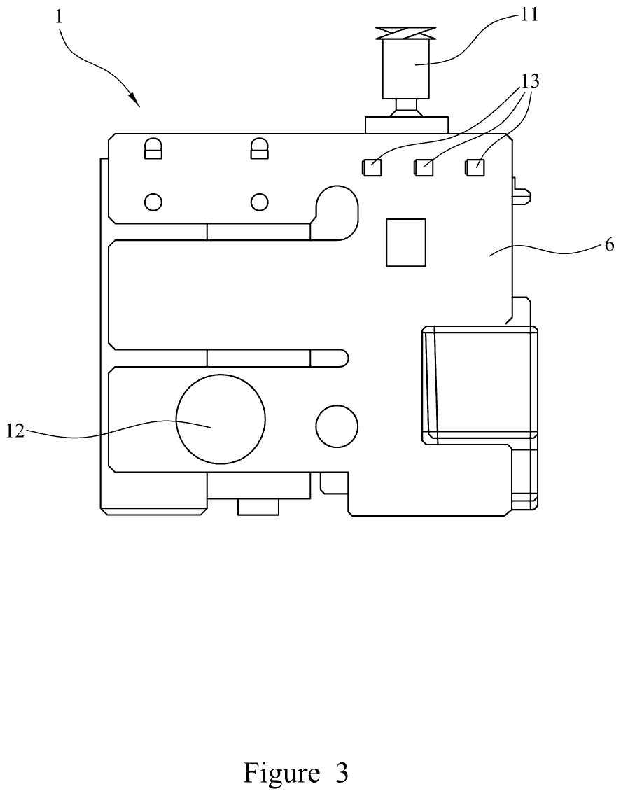

FIGS. 1-3 illustrate a device in accordance with the present invention.

A device (1) in accordance with the present invention comprises a vacuum pump (2), in this example contained within a manifold (3). A single gas reservoir (4) is connected to the pump (2) via a one-way valve (5). The vacuum pump (2) is secured to a printed circuit board (PCB) (6) comprising an electronic pressure sensor (7) corresponding with a branch (8) of the single gas reservoir (4). Battery terminals (9) are attached to the PCB (6) for accommodating batteries (10). A standard female Luer lock (11) extends from the single gas reservoir (4), for connection to a wound dressing for NPWT. A power button (12) and LED indicators (13) are located on the PCB (6), on the reverse side to the vacuum pump (2) and other components.

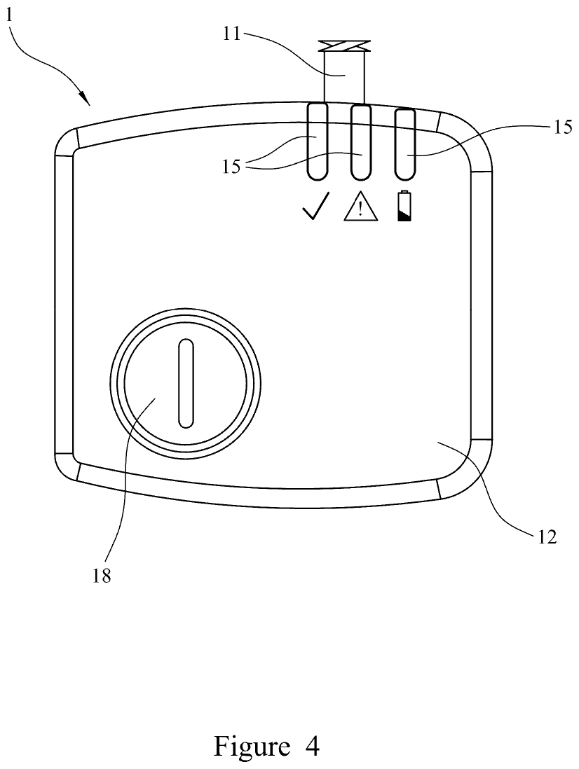

FIG. 4 is a front-view illustration of a housing (12) in accordance with the present invention. A button (18) on the housing corresponds with the power button (12) on the PCB (6) and indicator windows (15) correspond with the LED indicators (13) on the PCB (6).

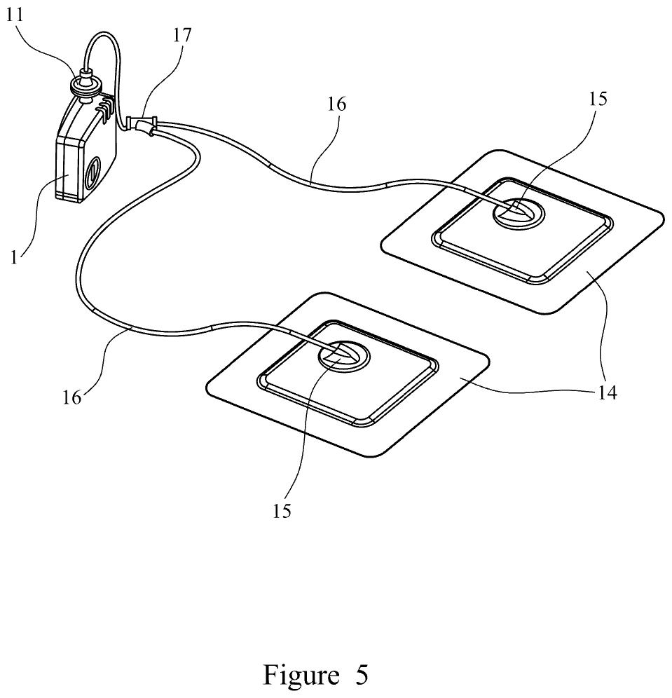

FIG. 5 illustrates a device (1) in accordance with the present invention, connected to two dressings for NPWT (14). An aperture (15) in each of the dressings (14) is connected to suction tubing (16) which is turn is connected with a Y-piece (17) that connects to tubing extending from the Luer Lock (11).

The illustrated device is battery operated, is for single patient use, and is disposable.

In use, one or more wound dressing(s) for NPWT, such as a wound dressing as described in WO2022049363, is applied over a wound to which negative pressure is to be supplied. One end of a suction tube is attached to the aperture or port on the dressing for NPWT, and the other end of the suction tube is attached to the Luer lock (11), to form a substantially airtight connection between the device (1) and the wound. The vacuum pump (2) is then activated by depression of the button (18) on the housing, and air is extracted from the single gas reservoir (4), generating a negative pressure therein and, via the suction tube, at the wound site. The presence of the single gas reservoir (4) has the effect of reducing the speed at which negative pressure is experienced at the wound site. The pressure sensor (7) detects the negative pressure in the single gas reservoir (4) and deactivates the pump when the negative pressure in the single gas reservoir (4) reaches a first predetermined value. The reduced speed of delivery of negative pressure to the wound site means that excess negative pressure is not applied to the wound in the time interval between the pressure sensor detecting that the negative pressure has reached the first predetermined value and the pump (2) being deactivated. Upon deactivation of the pump (2), the negative pressure in the single gas reservoir (4) will decrease. When the pressure sensor (7) detects that the negative pressure in the single gas reservoir (4) has decreased beyond a second predetermined value, the pump (2) is re-activated and negative pressure is again applied the wound. Thus, operation of the device (1) maintains the negative pressure that is applied to the wound at a substantially constant level throughout the period of operation.

LED indicators (13) on the PCB (6) detect the whether the device is operational (on) or non-operational (off); if a leak is detected in the system; and if battery charge is low; and emit light, visible through the indicator windows (15) on the housing (12), to indicate when the device is on, when there is a system leak, and when battery charge is low, so that the user can take appropriate action.

Pressure release in the system is available by disconnecting the pump (2) from the dressing(s) (14); pressure will be maintained via the one-way valve (5).

When the wound dressing for NPWT (14) reaches its absorptive capacity, the device (1) should be switched off and the dressing (14) detached from the suction tube (16). A new wound dressing for NPWT (14) may then be attached to the suction tube (16) if therapy is to be continued.

1. A device for negative pressure wound therapy (NPWT), comprising:
a source of negative pressure and
a single gas reservoir in fluid communication with the source of negative pressure and comprising means for establishing fluid communication with a wound.

2. A device for NPWT as claimed in claim 1, wherein the source of negative pressure is a vacuum pump, for example, a diaphragm vacuum pump.

3. A device for NPWT as claimed in claim 1, wherein the source of reduced pressure has a maximum dimension of 100 mm or less and/or occupies a volume of 10 cm³ or less.

4. A device for NPWT as claimed in claim 1, wherein the source of reduced pressure is housed within a manifold.

5. A device for NPWT as claimed in claim 1, wherein the single gas reservoir is in fluid communication with the source of reduced pressure via a vacuum port located on the source of reduced pressure.

6. A device for NPWT as claimed in claim 1, wherein a check valve is located between the source of reduced pressure and the single gas reservoir.

7. A device for NPWT as claimed in claim 6, wherein the check valve is a one-way valve that permits the flow of gas out of the single gas reservoir but that does not allow a flow of gas into the single gas reservoir.

8. A device for NPWT as claimed in claim 7, wherein the one-way valve is selected from a diaphragm valve, a ball and spring valve and a swing/tilt valve.

9. A device for NPWT as claimed in claim 1, wherein the means comprised by the single gas reservoir, for establishing fluid communication with a wound, is a medical connector.

10. A device for NPWT as claimed in claim 9, wherein the medical connector is a Luer lock.

11. A device for NPWT as claimed in claim 1, further comprising means for detecting the negative pressure that is applied to the wound.

12. A device for NPWT as claimed in claim 11, wherein the means for detecting the negative pressure that is applied to the wound is operably coupled to the source of reduced pressure.

13. A device for NPWT as claimed claim 12, wherein the device comprises a programmable computer processor and the means for detecting the negative pressure that is applied to the wound is operably coupled to the source of reduced pressure via the programmable computer processor.

14. A device for NPWT as claimed in claim 1, wherein the device is battery operated.

15. A device for NPWT as claimed in claim 1, wherein the device is for single-patient use.

16. A device for NPWT as claimed in claim 1, wherein the device is disposable.

17. A device for NPWT as claimed in claim 1, comprising a housing that accommodates the components of the device.

18. A device for NPWT as claimed in claim 17, wherein the housing comprises a battery compartment.

19. A system for negative pressure wound therapy (NPWT), comprising a device for NPWT as claimed in claim 1 and at least one wound dressing for NPWT.

20. A system for NPWT as claimed in claim 19, wherein the wound dressing for NPWT is an island wound dressing comprising a backing layer and a wound contacting layer, and an absorbent pad disposed between the backing layer and the wound contacting layer, the backing layer comprising an aperture which brings the absorbent pad into fluid communication with the exterior of the wound dressing.

21. A system for NPWT as claimed in claim 20, wherein the aperture is located directly above or on top of the absorbent pad.

22. A system for NWPT as claimed in claim 20, wherein the wound contacting layer is a layer of perforated silicone gel.

23. A system for NPWT as claimed in claim 20, comprising a moisture-permeable layer of pressure-sensitive adhesive, disposed between the backing layer and the absorbent pad, or between the absorbent pad and the wound contacting layer.

24. A kit comprising a device for negative pressure wound therapy (NPWT) as claimed in claim 1 and a plurality of wound dressings for NPWT.

25. A kit for NPWT as claimed in claim 24, wherein the plurality of wound dressings for NPWT comprise an island wound dressing comprising a backing layer and a wound contacting layer, and an absorbent pad disposed between the backing layer and the wound contacting layer, the backing layer comprising an aperture which brings the absorbent pad into fluid communication with the exterior of the wound dressing.

26. A kit for NPWT as claimed in claim 24, further comprising any of batteries; suction tubing; a connector such as a Y-piece, and sealing strips for attaching a dressing for NPWT to the skin of a patient.

27. A method of negative pressure wound therapy (NPWT), comprising:
applying a wound dressing for NPWT to a wound;
establishing fluid communication between the wound dressing and the single gas reservoir of the device as claimed in claim 1; and
activating the device.

28. A method of NPWT as claimed in claim 26, wherein the wound dressing for NPWT comprises an island wound dressing comprising a backing layer and a wound contacting layer, and an absorbent pad disposed between the backing layer and the wound contacting layer, the backing layer comprising an aperture which brings the absorbent pad into fluid communication with the exterior of the wound dressing.
